(19) United States
(12) Patent Application Publication
(10) Pub. No.: US 2020/0330678 A1
(43) Pub. Date: Oct. 22, 2020

(54) DEVICES, SYSTEMS, AND METHODS FOR DELIVERY OF SOLID FORMULATIONS

(71) Applicant: ABBOTT DIABETES CARE INC., Alameda, CA (US)

(72) Inventor: Udo HOSS, Castro Valley, CA (US)

(73) Assignee: Abbott Diabetes Care Inc., Alameda, CA (US)

(21) Appl. No.: 16/305,275

(22) PCT Filed: Jul. 23, 2018
(86) PCT No.: PCT/US2018/043240

§ 371 Date: Nov. 28, 2018

Related U.S. Application Data
(60) Provisional application No. 62/535,314, filed on Jul. 21, 2017.

(51) Int. Cl. A61M 5/142; A61M 5/172; A61K 38/28; A61K 9/00

(52) U.S. Cl. CPC A61M 5/14248; A61M 5/1723; A61K 38/28; A61K 9/0024; A61M 2005/14252; A61M 2205/18; A61M 2205/50; A61M 2205/3303; A61M 2205/502; A61M 2205/3553; A61M 2205/3306

(57) ABSTRACT

The disclosed technology provides delivery devices for therapeutic agents, where the delivery devices are configured to be worn on a patient's body, and where the delivery devices deliver a solid formulation of a therapeutic agent subcutaneously to the patient. The delivery devices may in one embodiment be part of an integrated system comprising both a solid formulation delivery device and an analyte sensing device.

### CROSS-REFERENCE TO RELATED APPLICATIONS

This application claims priority to U.S. provisional application 62/535,314, filed on Jul. 21, 2017.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Certain medical conditions or diseases require that patients receive a drug or therapeutic agent multiple times daily for control of certain aspects of the medical condition or disease. For example, in the treatment of diabetes, dosages of insulin can be administered subcutaneously either by injection or delivered via a pump several times daily to control glucose levels.

Conventional on-body liquid insulin delivery systems, which generally use tethered or adhesive patch pump devices, use a partially implanted cannula or catheter for administration of the insulin to the patient. Due to the risk of infection, inflammation, and irritation, these cannulas or catheters need to be replaced every two to three days, and the site of insertion needs to be rotated to reduce the severity of adverse reactions. Further, it is believed the adverse reactions observed with the use of the continuous liquid insulin delivery systems are due to the constant infusion of liquid into tissue, since partially implanted cannulas and sensors—such as those used with sensor device systems that do not deliver insulin—can remain in the same location on the patient for longer periods without risking the adverse reactions described above. Thus, a pump system that delivers therapeutic agents in solid form would be beneficial.

Additionally, for many proteins and peptides of moderate molecular weights such as insulin, a solid formulation stored under dry conditions is generally preferable and more stable than storage in a liquid state. Although methods to manufacture dry powder insulin from a liquid state are known in the medical field, reliable and economic methods have been lacking for delivering the dry powder to a user in a way that ensures a consistent and cost-effective systemic delivery.

It is thus desirable in the art to have an on-body delivery device to address the current need for delivery systems for solid therapeutics to overcome the above-described disadvantages.

### BRIEF DESCRIPTION OF THE DRAWINGS

FIG. 1 is a simplified schematic diagram showing an embodiment of a solid formulation delivery device (“delivery device”) including a housing containing components to dispense solid therapeutic particles.

FIG. 2A is a cross-sectional view of a delivery device with a piston in a fully deployed mode.

FIG. 2B is a cross-sectional view of a delivery device with a piston in a retracted mode showing an early stage of delivery of a solid therapeutic particle.

FIG. 2C is a cross-sectional view of a delivery device with a piston in a delivery mode in a mid-stage of delivery of a solid therapeutic particle.

FIG. 2D is a cross-sectional view of a delivery device with a piston in a delivery mode showing the delivery of a solid therapeutic particle to the subcutaneous tissue of a patient.

FIG. 3 is a cross-sectional view of another embodiment of a delivery device with a piston in a fully deployed mode showing the delivery of a single solid therapeutic particle.

FIG. 4 is a cross-sectional view of another embodiment of a delivery device with a piston in a fully deployed mode showing the delivery of two solid therapeutic particles.

FIG. 5A illustrates additional embodiments for filling or refilling a formulation reservoir while a delivery device is positioned on a patient.

FIG. 5B illustrates additional embodiments for filling or refilling a formulation reservoir while a delivery device is positioned on a patient.

FIG. 6 is a schematic diagram showing an example of an integrated system and various data processing/storage devices.

FIG. 7 is a schematic diagram showing a side cross-sectional view of an integrated system that includes a delivery device, an analyte sensing device, and a reader.

FIG. 8 is a schematic diagram showing a side cross-sectional view of an integrated system that includes a delivery device, an analyte sensing device, and a reader.

FIG. 9 is a schematic diagram showing a side cross-sectional view of an integrated system that includes a delivery device, an analyte sensing device, and a reader.

FIG. 10 is a block diagram of a data processing unit from an integrated system that includes both a solid formulation delivery device and an analyte sensing device.

FIG. 11 is a schematic diagram showing a side cross-sectional view of an integrated system that includes a delivery device, an analyte sensing device, and a reader.

FIG. 12 is a schematic diagram showing a side cross-sectional view of an integrated system that includes a delivery device, an analyte sensing device, and a reader.

FIG. 13 is a cross sectional view of another example of a delivery device according to the disclosed technology.

FIG. 14 is a cross sectional view of another example of a delivery device according to the disclosed technology.

### DESCRIPTION

The following descriptions and embodiments are put forth so as to provide those of ordinary skill in the art with a complete disclosure and description of how to make and use the disclosed devices, systems, and methods, and are not intended to limit the scope of the disclosed devices, systems and methods. It will be appreciated by persons skilled in the art that numerous variations and/or modifications may be made to the disclosed devices, systems and methods as shown in the specific embodiments without departing from the spirit or scope of the disclosed devices, systems and methods as broadly described. The present embodiments are, therefore, to be considered in all respects as illustrative and not restrictive.

Note that as used herein and in the appended claims, the singular forms “a,” “an,” and “the” include plural referents unless the context clearly dictates otherwise. Thus, for example, reference to “therapeutic agent” refers to one or more such agents, and reference to “the system” includes reference to equivalent means and components known to those skilled in the art, and so forth.

Unless defined otherwise, all technical and scientific terms used herein have the same meaning as commonly understood by one of ordinary skill in the art to which this disclosed technology relates. All publications mentioned herein are incorporated by reference for the purpose of describing and disclosing devices, systems and methods that may be used in connection with the disclosed devices, systems and methods.

Where a range of values is provided, it is understood that each intervening value, between the upper and lower limit of that range and any other stated or intervening value in that stated range is encompassed. The upper and lower limits of these smaller ranges may independently be included in smaller ranges, and are also encompassed, subject to any specifically excluded limit in the stated range. Where the stated range includes one or both of the limits, ranges excluding either both of those included limits are also included.

In the following description, numerous specific details are set forth to provide a more thorough understanding of the devices, systems and methods. However, it will be apparent to one of skill in the art that the devices, systems and methods may be practiced without one or more of these specific details. In other instances, features and procedures well known to those skilled in the art have not been described in order to avoid obscuring the disclosed devices and methods.

The current disclosure includes systems, devices, apparatus, and methods that may be used to provide delivery of solid therapeutic particles to a patient. Continuous infusion of liquid medications can generate a number of adverse reactions in patients including inflammation, irritation, and increased risk of infection at the infusion site, possibly related to the constant infusion of liquid into tissue. Additionally, precise and repeatable measurement and delivery of liquid medications presents other problems. Some liquid medications can separate and/or settle under certain conditions leading to potential variations in concentration and dosing. Air bubbles, leakage at seals or gaskets, and other issues may cause inaccurate dosing volume. Solid medications, in contrast, are more stable, may generate less irritation at the insertion site, and more easily produce an accurate and repeatable dose of medication. Solid medications are also more easily formulated for controlled-release over time.

Delivery devices of the disclosed technology may be used alone, or may be configured as part of an integrated system to monitor analyte level(s) within a patient's body and provide therapy recommendations. Such integrated system may include delivery devices and any or all of the following components: an on-body analyte sensing device (“sensing device”), a remote reader and/or smart phone in electronic communication with the delivery device and/or sensing device, a local computer in electronic communication with remote reader and/or smart phone as well as one or more remote servers. The delivery device may be placed on a patient's body with an inserter.

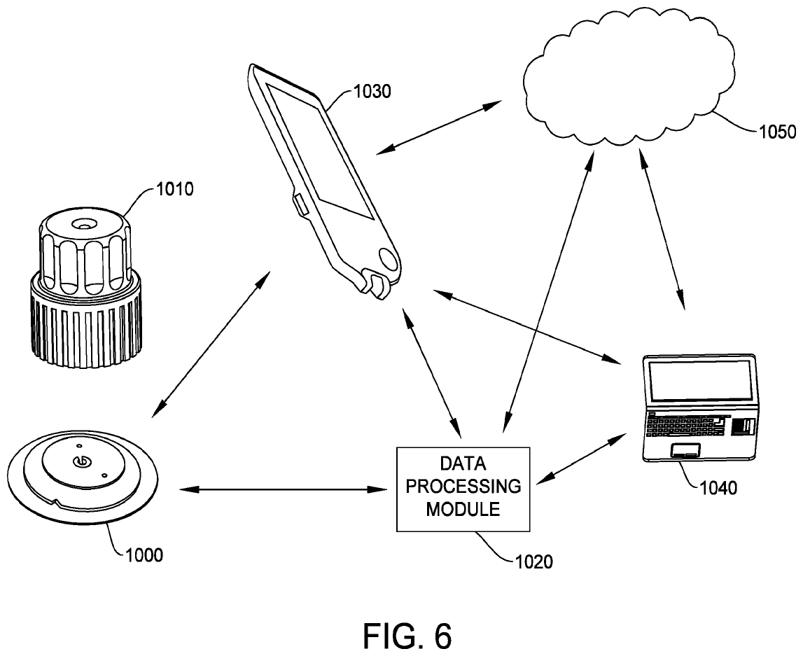

One such example of an inserter is shown in FIG. 6. In this particular example, an integrated on-body housing unit 1000 includes both a solid formulation delivery device and an analyte sensing device having an adhesive layer for attachment on the skin of a user. An insertion device 1010 may be used to transcutaneously position a portion of the on-body unit 1000 through the user's skin surface and in contact with bodily fluid as well as to position the adhesive layer on the unit on the skin surface. One example of such an inserter unit is described in U.S. Pat. No. 9,215,992, the disclosure of which is incorporated in full and for all purposes herein by reference.

Insertion of devices according to the disclosed technology in a user will vary according to the specific construction and configuration of a particular device. For example, insertion and placement of an on-body unit such as shown in FIG. 6 using an insertion device might begin with the unit disposed within the insertion device and held in position by a releasable friction fit. A carriage needle/sharp on the insertion device is positioned through a septum in the on-body unit. The combined unit is brought into contact with the user's skin at the desired insertion point. Depression of an insertion device actuator causes longitudinal movement of the sharp towards the surface of the user's skin. As the sharp is urged distal from the inserter, it carries the cannula of the on-body unit into the subcutaneous portion of the user's skin. Once the user stops providing a downward force on the inserter a spring biases the inserter upward and withdraws the sharp from the user's skin. The inserter device can then be removed leaving the on-body unit on the user, typically secured using an adhesive surface, and the cannula of the unit transcutaneously placed.

The delivery device may include a housing containing a formulation reservoir configured to contain solid therapeutic particles, a solid therapeutic particle distribution mechanism within the formulation reservoir, a particle detection system to detect delivery of the solid therapeutic particles, a cannula, a piston channel, a delivery channel connected to the cannula and to the piston channel, a piston that can be extended and retracted within the piston channel and into the delivery channel and cannula, a piston drive mechanism that controls the deployment and retraction of the piston, and a processor-controller apparatus that controls the piston drive mechanism in the delivery device such that a precise number of solid therapeutic particles are delivered.

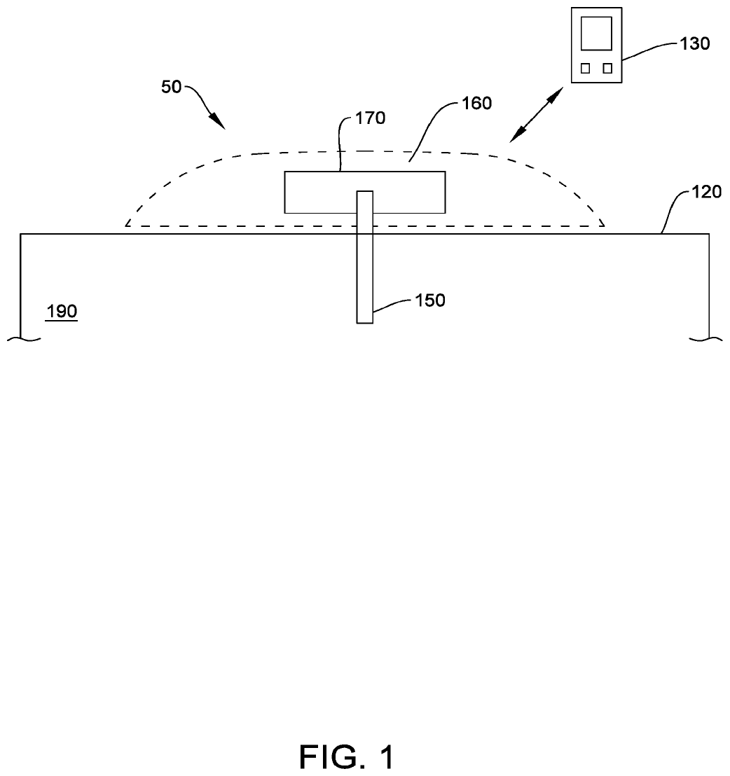

FIG. 1 shows a simplified solid formulation delivery device 50. Delivery device 50 may include a housing 160 that can be adhered to a user's skin 120, a formulation reservoir 170, and a cannula 150 for delivery of solid therapeutic particles to the subcutaneous tissue 190 of a patient.

The insertion depth of the cannula can be anywhere from 2 mm to 12 mm. Different embodiments include insertion depths of, e.g., 3-9 mm and 5-7 mm. The insertion depth can be selected based on variables such as size of the delivery device, the particle size, the delivery amount of the therapeutic agent per particle, frequency of delivery, patient age and weight, and the like. The inner diameter or cross-section of a cannula for delivery of solid therapeutic particles typically is from 50 μm to 500 μm, encompassing any value and subset therebetween, including 100 μm to 350 μm, and 150 μm to 250 μm. Accordingly, the diameter of the solid therapeutic particles of the present disclosure may be any size that is less than the inner diameter or cross-section of the cannula. In some embodiments, the diameter of the solid therapeutic particles may, for example, be in the range of less than 50 μm to less than 500 μm, encompassing any value and subset therebetween, including less than 100 μm to less than 350 μm, and less than 150 μm to less than 250 μm. Cannulae for use with the disclosed delivery devices (and disclosed integrated delivery devices) may include a variety of suitable materials such as stainless steel, polymers, silicone, and the like. Cannulae made from sufficiently ridged material, such as metal, may act as a sharp to pierce the user's skin during insertion and remove the need for a separate sharp or needle. Cannulae made from flexible materials such as silicone made cause less irritation at the insertion site. Such cannulae may also be designed to retract or collapse after passage of a solid therapeutic particle thereby preventing such particles from travelling back up the cannula after injection into the user.

The housing may be a single housing component 160 as shown in FIG. 1 or optionally may include two or more housing components. One or more optical elements can be molded into the housing of the on-body delivery device to magnify an area or areas of interest. For example, in some embodiments, the housing may be equipped with a window that permits a user to determine qualitatively or quantitatively the remaining amount of solid therapeutic particles in the formulation reservoir of the delivery devices disclosed herein. An optical element, such as a lens or light source, may be molded or otherwise affixed to the housing in order to magnify the window area to allow a user to easily view the depletion of the solid therapeutic agents. In some embodiments, for example, the window may be display a colorimetric indicator that can be magnified with the optical element, where the indicator shows a sort of “count down” for the depletion of the solid therapeutic particles and more or less color along a scale of the indicator indicates depletion. In an embodiment where the housing includes more than one housing component, the two or more components of the housing may be entirely separate from each other, or components of the housing may be connected together, for example, by a hinge, to facilitate the coupling of the components to form the housing. Two or more separate components of the housing may have complementary, interlocking structures, such as, for example, interlocking ridges or a ridge on one component and a complementary groove on another component, or snap-fit features so that the two or more separate components may be easily and/or firmly coupled together. This may be useful, particularly if the components are taken apart and fit together occasionally, for example, when a power supply or formulation reservoir is replaced. Other fasteners may also be used to couple the two or more components together, including, for example, screws, nuts and bolts, nails, staples, rivets, or the like. In addition, adhesives, both permanent and temporary, may be used including, for example, contact adhesives, pressure sensitive adhesives, glues, epoxies, adhesive resins, and the like. Typically, the housing is at least water resistant to prevent the flow of fluids into contact with the components in the housing, including, for example, conductive contacts, and in some embodiments, the housing is waterproof.

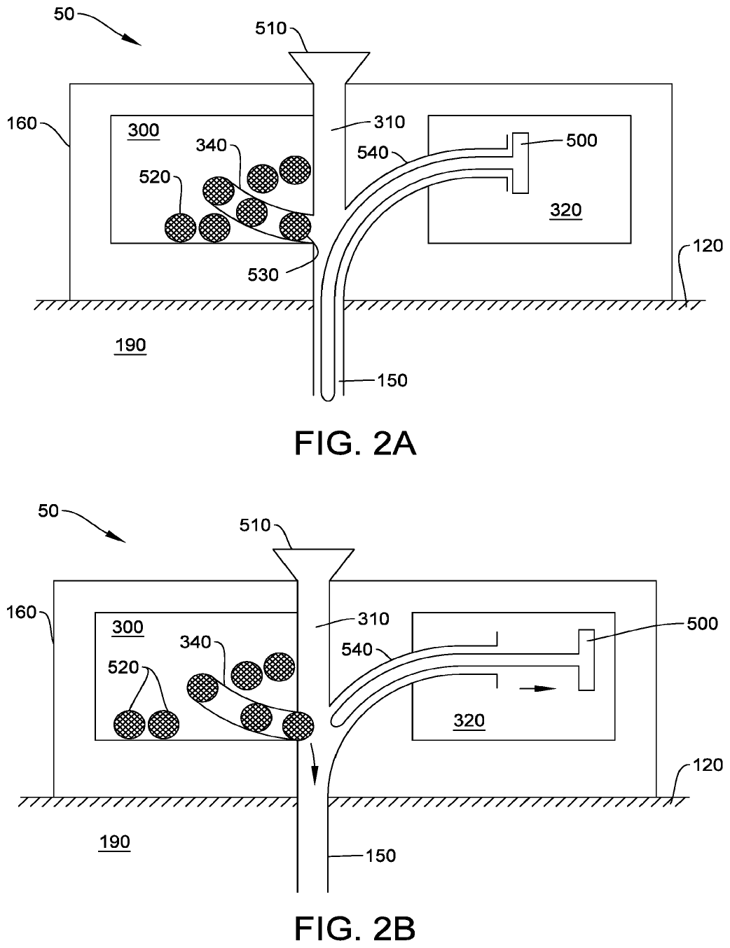

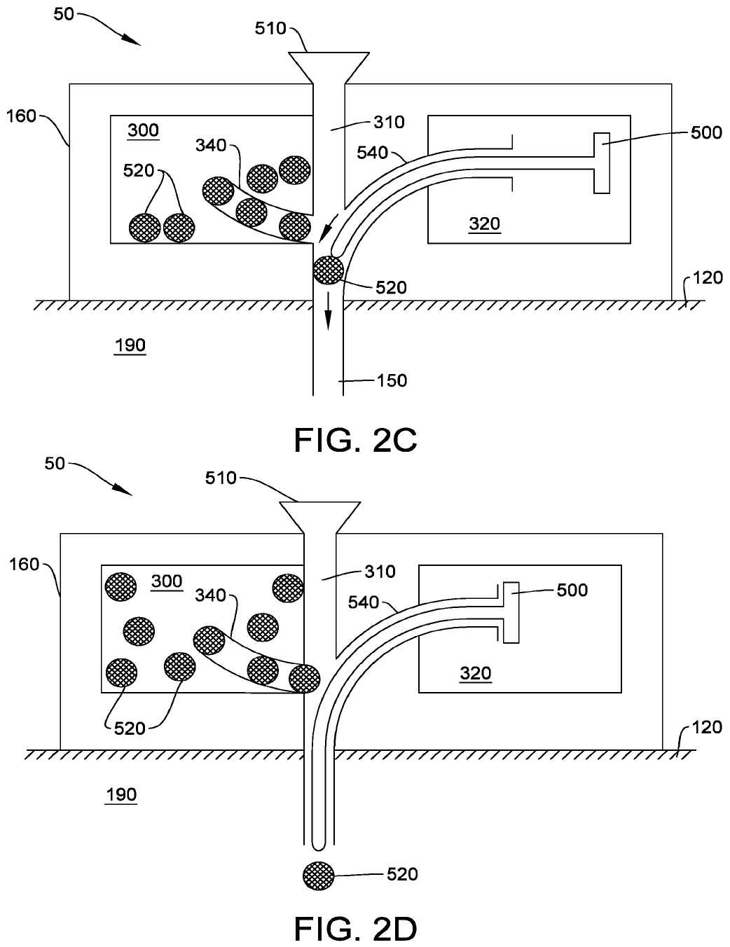

FIGS. 2A, 2B, 2C, and 2D are cross sectional views illustrating an example of a delivery device 50 including a housing 160 containing a formulation reservoir 300, a delivery channel 310 having a septum 510 at the proximal end of the delivery channel 310 and a cannula 150 at a distal end of a delivery channel 310. Distal end of cannula 150 is placed into the subcutaneous tissue 190 of the patient. The septum 510 may be self-sealing after a sharp (not shown) has been inserted through the septum then removed during introduction of the delivery device onto the skin of a patient. Delivery device 50 uses a piston 500 as a transfer mechanism for pushing solid therapeutic particles 520 through cannula 150 and into the patient. Therapeutic formulation particles 520 enter the delivery channel 310 from a solid therapeutic particle distribution mechanism 340 in formulation reservoir 300 through reservoir port 530 upon retraction of piston 500 into piston channel 540. Subsequent advancement of piston 500 through piston channel 540 and into delivery channel 310 forces the solid therapeutic particles through delivery channel 310 and through cannula 150. The positioning of piston 500 is controlled by a piston drive mechanism 320 in engagement with piston 500 and may be controlled by a processor-controller apparatus (not shown).

Formulation reservoir 300 of delivery device 50 may include a solid therapeutic particle distribution mechanism 340 within formulation reservoir 300 that allows for a controlled exit of a defined number of solid therapeutic particles through the reservoir port 530 of formulation reservoir 300. The solid therapeutic particle distribution mechanism 340 assembles or presents the solid therapeutic particles in a coordinated fashion for delivery into the delivery channel. The particle distribution mechanism may be of any configuration as long as a defined number of solid therapeutic particles are presented for delivery by engagement with the piston in a reliable, reproducible manner. For example, the solid therapeutic particles may be packaged in a “blister pack” and arranged as a linear belt of encapsulated solid therapeutic particles. The piston, when presented with an encased solid therapeutic particle, pushes the solid therapeutic particle out of the encasement and into the delivery channel. Such a mechanism may include an actuator that advances the linear belt of encapsulated solid therapeutic particles from the formulation reservoir through the reservoir port for engagement with the piston in the delivery channel, and re-winds the empty encasements back into, e.g., the formulation reservoir. In another embodiment, solid particles are arranged in a cartridge-type configuration such that only a single particle may be dispensed at a time. In yet another embodiment, a particle distribution mechanism is not used, and the timing of the retraction and deployment of the piston is such that only one solid therapeutic particle exits through the reservoir port 530 at a time. In some embodiments, the solid therapeutic particles may be coated to facilitate their entry into and exit from the cannula. For example, the solid therapeutic particles may be at least partially coated with an anti-stick compound or anti-friction compound to aid in their traversal through the cannula and into a biological fluid.

FIG. 2A shows piston 500 in full extension (or deployment) through piston channel 540, delivery channel 310 and cannula 150. FIG. 2B shows piston 500 retracted through piston channel 540 further up into delivery channel 310 and out of cannula 150, with a solid therapeutic particle 520 exiting formulation reservoir port 530. FIG. 2C shows piston 500 being deployed once again through piston channel 540 back down into delivery channel 310 pushing a solid therapeutic particle 520 through delivery channel 310 and into cannula 150. FIG. 2D shows solid therapeutic particle 520 delivered from cannula 150 to the subcutaneous tissue 190 of a patient.

In some embodiments, the delivery device further comprises mechanisms to prevent the solid therapeutic particles from reentering the delivery device or bodily fluids from entering the delivery channel and, in particular, the formulation reservoir. For example, as mentioned previously, the piston may be deployed in the delivery channel and cannula at all times except when a solid therapeutic particle is exiting the formulation reservoir and entering the delivery channel. In another example, the cannula may be made of a collapsible material such as silicone or some other suitable material. Generally, the collapsible material may be any material that is sufficiently flexible to allow the cannula to collapse and prevent backflow of the solid therapeutic particles back into the delivery device. In some embodiments, such materials have mechanical properties identical or similar to silicone. In other embodiments, the collapsible cannula may be made of an inner and outer material, where the outer material is rigid and the inner material collapses as a whole, or has one or more independent valves that close with the piston is in the cannula and open to bloc all or a portion of the cannula when the piston is removed from the cannula, thereby preventing backflow of the solid therapeutic agents.

In some embodiments, therapeutic agents are formulated into hydrophilic particles that rapidly and easily dissolve in subcutaneous tissue following delivery, allowing for the solid therapeutic particles to be administered into the subcutaneous tissue without need for prior solubilization. For example, such hydrophilic particles may be formed by attaching, bonding, mixing, coating, or otherwise affiliating a hydrophilic group and/or compound to the solid therapeutic particles described herein. Any hydrophilic group may be suitable, provided that it is biocompatible and biodegradable and does not substantially interfere with the efficacy of the solid therapeutic particles. For example, such hydrophilic groups and/or compounds may include, but are not limited to, a polyethylene glycol (PEG), one or more sugar groups (e.g., glucose, mannose, sucrose, and the like), a hydrophilically-modified polylactic acid (PLA), hydrophilic hydrogels, a starch, and the like, and any combination thereof. The solid therapeutic formulations that can be delivered by the disclosed delivery device include any agents that can be successfully delivered subcutaneously. Exemplary agents that can be delivered include proteins, peptides, antigens and low molecular weight drugs, including, e.g., insulin, lipid regulators, antidepressants, narcotic analgesics, beta-blockers, ACE inhibitors, anti-rheumatics, sedatives, antibiotics, and the like. In some embodiments, the solid therapeutic particles may be at least partially coated or encapsulated with one or more compounds that change the release profile and/or biological impact of the therapeutic agent. For example, the compound may be a degradable material that allows the agent to slowly release, or release at known time intervals. Alternatively or in addition, the compound may be used to alter the biological impact of the agent, such as to increase its biological activity, decrease its biological activity, increase the agent's rate of absorption into a biological fluid or tissue, decrease the agent's rate of absorption into a biological fluid or tissue, and the like, and any combination thereof. In a non-limiting example, for instance, the compound may be hyaluronidase, which may increase the biological absorption of an insulin agent.

In some embodiments, the particles are formulated so that each solid therapeutic particle provides a predictable and tightly-controlled dose of a therapeutic agent, and the delivery device administers a highly-regulated dose of the solid therapeutic particles to a targeted site of the patient. In some embodiments, the solid therapeutic particles have a specific hydrophilicity so that they are easily dissolvable in the subcutaneous tissue. The solid therapeutic particles may also comprise one or more conventional pharmaceutical carriers, adjuvants, diluents and/or excipients appropriate for formulating a solid therapeutic particle, where, optionally, such carriers, adjuvants, diluents and/or excipients enhance the dissolvability of the solid therapeutic particles. The shape and surface properties of the solid therapeutic particles may also be modified to ensure efficient delivery and dissolvability, e.g., the solid therapeutic particles can be porous or the surface thereof modified to prevent aggregation in the reservoir. In some embodiments, the solid therapeutic particles are spherical. In some embodiments, the solid therapeutic particles are substantially spherical (e.g., having a spherical or elliptical geometry, including irregular spheres, ellipsoids, ovoids, platelets, capsules, and the like), non-spherical, or polygonal. For example, in some embodiments, the solid therapeutic particles may be filament shaped (e.g., thread-like), cylindrical, conical, or any other shape capable of flowing through the cannula of the delivery devices described herein when the cannula is not collapsed. Embodiments include solid therapeutic particles that are of uniform size to ensure a predictable administration of the desired dosage. In certain embodiments, the solid therapeutic particles are exposed to an agent that prevents aggregation (e.g., a desiccant) while in the formulation reservoir, and in some embodiments, the formulation reservoir is hermetically sealed before use and contains a desiccant to ensure the solid therapeutic particles remain dehydrated. The desiccant may sorb moisture by physical or chemical means. Examples of such desiccants may include, but are not limited to, silicon, silica, silicon dioxide, a clay (e.g., bentonite), calcium oxide, a molecular sieve, carbon, and the like, and any combination thereof. The solid therapeutic particles may be exposed to the desiccant by any means capable of prevent aggregation thereof and/or sorption of moisture including, but not limited to, the desiccant being molded within at least a portion of the formulation reservoir (e.g., to an interior wall), the desiccant being attached or otherwise coated onto at least a portion of the formulation reservoir, the desiccant being free-floating within the formulation reservoir but shaped or sized such that it is unable to travel into the cannula, and the like, and any combination thereof.

In some embodiments, the therapeutic formulation particles are a solid (e.g., crystalline) formulation of insulin or an insulin analog. The insulin is formulated into solid therapeutic particles that are relatively uniform in size and dimension and are configured for presentation by the particle delivery mechanism to the piston for delivery through the delivery channel and cannula of the delivery device. For basal administration, an insulin basal rate may be established through a combination of a specific concentration of insulin per particle and control of the number of particles released. Bolus administration may be established in the same way or may be established using a solid therapeutic particle with a greater concentration of insulin; e.g., contained in separate reservoirs as shown in FIG. 4. The concentration of insulin or insulin analog in solid therapeutic particles used for basal administration is preferably 0.05 to 1.0 unit/particle. For example, for a basal infusion—depending on the individual patient—a dose may be from 0.05 units per hour to 3.0 units per hour, or 0.05 units per hour to 2.0 units per hour, such as for a total delivery of 1.2 units to 72 units of insulin per day, or a total delivery of 12 units to 48 units of insulin per day, encompassing any value and subset therebetween. The frequency of delivery may be 1× per hour, or 2× per hour, or 3×, 4×, or more per hour. The more frequent the delivery, the more likely the basal level of the therapeutic agent will be smooth; that is, the level of therapeutic agent will not vary greatly from low to high concentration in the individual patient. For example, for a 24 unit delivery over 24 hours, the delivery may be 1 unit of insulin per hour, delivered at 0.25 units every 15 minutes. Bolus delivery may be achieved using a greater loading or more frequent delivery (e.g., rate of delivery) of any of the particle doses described with reference to basal delivery (e.g., 0.05 to 1.0 unit/particle). Alternatively or in combination, bolus delivery may include particles themselves having a greater concentration of insulin, or alternatively may be larger in size. For example, in some embodiments where the solid therapeutic particle is a filament, a bolus dose may be achieved by addition of a longer filament and, therefore, a greater concentration of insulin. Moreover, the delivery device described herein may be only a basal delivery device, only a bolus delivery device, or a combination of a basal and bolus delivery device, without departing from the scope of the present disclosure.

Further the insulin particles may comprise various insulin analogs that have absorption properties that are tailored for particular uses or dosage regimes. Examples of fast-acting insulin analogs include insulin lispro (Humalog™), B28Asp human insulin (NovoLog™ or NovoRapid™), and insulin glulisine (Apidra™). Examples of long-acting insulin analogs include ultralente, insulin demetir (Levemir™), insulin degludec (Tresiba™) and insulin glargine (Lantus™). An insulin analog with delayed absorption after subcutaneous introduction, such as NPH insulin, may also be used.

In some embodiments, the delivery device may not include a power source, or on-board electronics. Such a delivery device provides the advantage of being inexpensive and durable. In such a configuration, a button may be operably connected to the piston drive mechanism so that the user can initiate administration of the solid therapeutic particle from the formulation reservoir. The button operably advances the piston through the piston channel, into the delivery channel, and through the cannula to deliver solid therapeutic particles to the patient. In addition, the delivery device or portions thereof may be transparent, such that a user can confirm delivery of the solid therapeutic particles.

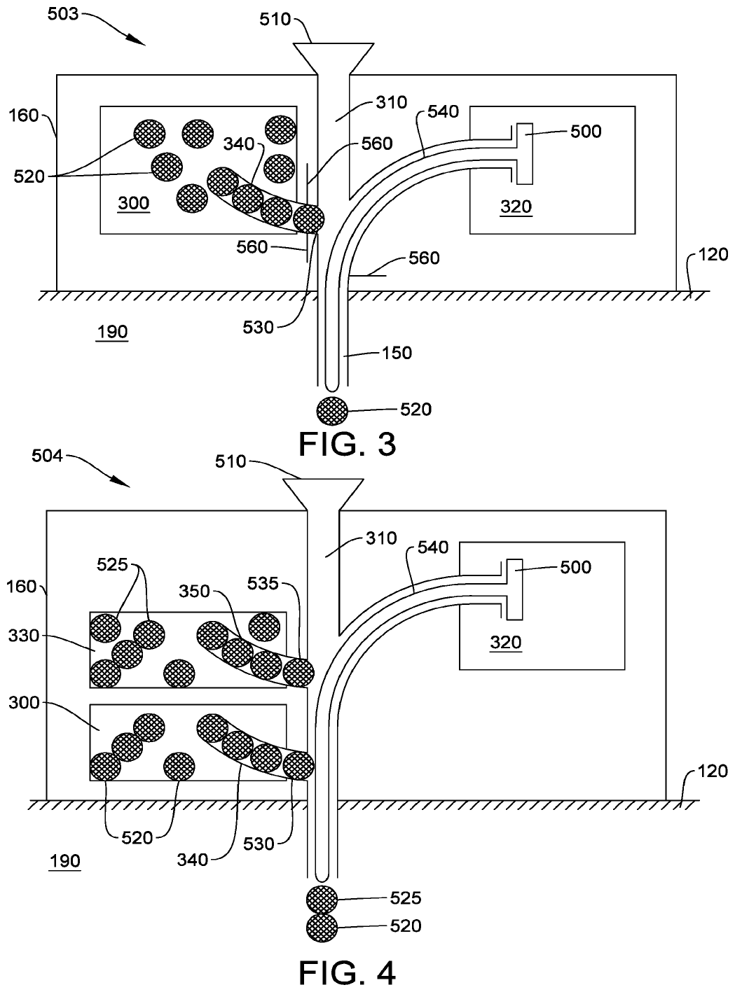

FIG. 3 is a cross sectional view of an embodiment of a delivery device 503 including two particle detection mechanisms 560, an external housing 160 containing a formulation reservoir 300, a delivery channel 310 and a cannula 150 that is placed into the subcutaneous tissue 190 of a patient. A proximal end of cannula 150 is coupled to a distal end of delivery channel 310, and a distal end of cannula 150 is inserted into the patient. Delivery device 503 uses a piston 500 for expelling solid therapeutic particles 520 through cannula 150 and into the patient. The solid therapeutic particles 520 enter delivery channel 310 via the particle distribution mechanism 340 through reservoir port 530 upon retraction of piston 500 into piston channel 540. Advancement of piston 500 through piston channel 540 into delivery channel 310 forces solid therapeutic particle 520 into delivery channel 310, through the cannula 150, to be delivered to the subcutaneous tissue 190 of the patient. The positioning of piston 150 is controlled by a piston drive mechanism 320 in engagement with piston 500 and controlled by a processor-controller apparatus (not shown).

As seen in FIG. 3, in some embodiments, the release and movement of the solid therapeutic particles may be confirmed through one or more particle detection mechanisms. A particle detection mechanism may be an optical or mechanical detection mechanism. In some embodiments, a particle detection mechanism tracks or counts each solid therapeutic particle that is delivered and in some embodiments confirms delivery of the solid therapeutic particles to the cannula. Additionally, an optical or mechanical particle detection mechanism may work with the processor-controller apparatus to keep track of the amount of solid therapeutic particles remaining in the formulation reservoir. For example, after a solid therapeutic particle is dispensed, the processor-controller apparatus may automatically reduce the number of known stored solid therapeutic particles by the number of dispensed solid therapeutic particles. In some embodiments, the particle detection mechanism may detect any blockages or occlusions of the delivery of solid therapeutic particles, e.g., by monitoring movement of the solid therapeutic particle into the delivery channel, and/or by monitoring movement of the piston. Additionally, the delivery device may include a mechanical detection mechanism to monitor piston pressure within the delivery channel or cannula in order to detect any blockages or occlusions of the delivery of solid therapeutic particles. The delivery device may provide an alarm to alert the user of an occlusion or failure of proper delivery of a solid therapeutic particle.

Two particle detection mechanisms 560 are shown in FIG. 3. One particle detection mechanism 560 is shown at the junction between particle distribution mechanism 340 and reservoir port 530, which is employed to ensure presentation of solid therapeutic particles to the piston by the particle distribution mechanism. The other particle detection mechanism 560 is shown at the junction between delivery channel 310 and cannula 150, which is employed to ensure delivery of solid therapeutic particles by the piston to the cannula. Particle detection mechanisms may include optical, mechanical, electrical, and combinations thereof. For example, an optical detection type system in a system having a blister pack style storage of solid therapeutic particles may attempt to pass a beam of light through the blister material at a point after which the therapeutic particle is to be distributed. If the light passes through then the system knows the particle was correctly distributed. If the light is blocked, then the system knows that the particle still occupies the blister and was not correctly distributed. In another example, an optical detection system may be configured to detect passage of objects through a particular point in a delivery channel or cannula, such as by breaking a beam of light or detecting the shadow of a passing object. If the system fails to detect passage of an object prior to detecting the insertion mechanism (such as a piston or the like) then the system knows that a therapeutic particle was not properly inserted.

In some embodiments, the delivery device may include two or more formulation reservoirs, each with a different solid therapeutic agent or formulation of a solid therapeutic agent. The delivery devices are designed such that the piston mechanism can be used to either deliver the solid therapeutic particles from a lower chamber or chambers of the delivery device, deliver solid therapeutic particles from an upper chamber or chambers of the delivery device, or to deliver solid therapeutic particles from both bottom formulation reservoir(s) and upper formulation reservoir(s). The deployment/retraction length of the piston and the particle distribution mechanisms in each formulation reservoir control from which formulation reservoir or reservoirs solid therapeutic particles are delivered.

FIG. 4 is a cross-sectional view of an exemplary delivery device 504 including an external housing 160 with a first (lower) formulation reservoir 300 and a second (upper) formulation reservoir 330 in contact with reservoir ports 530, 535. The formulation reservoirs 300, 330 contain particle distribution mechanisms 340, 350 for dispensing solid therapeutic particles 520, 525. Delivery device 504 includes a piston 500 for expelling solid therapeutic particles 520, 525 presented for delivery via particle distribution mechanisms 340, 350 through reservoir ports 530, 535 to delivery channel 310 and into cannula 150, which is placed in the subcutaneous tissue 190 of the patient. The external housing 160 further includes a piston drive mechanism 320 controlled by a processor-controller apparatus (not shown) that engages with and controls the position of piston 500. The delivery device 504 optionally comprises a septum 510 at the proximal end of delivery channel 310 for introduction of delivery device 50 by a sharp (not shown). Cannula 150 projects from the distal end of delivery channel 310 and into the patient. Delivery device 504 uses a piston 500 for expelling the solid therapeutic particles 520, 525 through cannula 150 and into the patient. The solid therapeutic particles enter delivery channel 310 through reservoir ports 530, 535 upon retraction of piston 500. Subsequent advancement of piston 500 into delivery channel 310 forces the solid therapeutic particles 520, 525 into delivery channel 310, and through cannula 150 into the subcutaneous tissue 190 of the patient.

The extent of deployment/retraction of piston 150 and presentation of solid therapeutic particles via particle distribution mechanisms 340, 350 controls the delivery of solid therapeutic particles 520, 525 from first and second formulation reservoirs 300, 330. Positioning of piston 500 through piston channel 540, delivery channel 310, and cannula 150 is controlled by a processor-controller apparatus that controls piston drive mechanism 320 in engagement with piston 500. Particle detection mechanisms optionally are employed in double or multiple reservoir delivery devices (see FIG. 3). Note that FIG. 4 shows an embodiment of a double reservoir delivery device where the formulation reservoirs are configured as upper and lower formulation reservoirs. It should be understood, however, that any number and configuration of formulation reservoirs may be used; e.g., side-by-side, axially arranged around the delivery channel, two sets of upper and lower formulation reservoirs, and the like.

In alternative embodiments of a double or multiple reservoir delivery device, the bottom reservoir may contain insulin for maintenance of the basal insulin level, and the top chamber may contain a glucose level modifying agent such as glucagon to counteract the insulin in the event of higher than desired levels of insulin in the patient. Such an embodiment of the disclosed technology would allow for use of solid glucagon which may be more stable, have a longer shelf life, and be easier to deliver accurately than liquid glucagon. Additionally, liquid glucagon is typically fast acting whereas solid preparations may include additional agents which delay release of the glucagon over time, if desired.

In still other embodiments, a multiple-reservoir device may contain therapeutic particles having different concentrations of the same medicant. For example, a two-reservoir device might contain particles which represent 5 units of insulin in one reservoir and particles which represent 1 unit of insulin in a second reservoir. Once the desired dosing level has been determined by a processor-controller device, the controller may also determine exactly how to formulate that dose. To provide a dose of 8 units of insulin, the controller might recommend one 5 unit particle and three 1 unit particles. If no 5 unit particles were available, the controller might recommend a dose of eight 1 unit particles and alert the user that the 5 unit particle reservoir was empty.

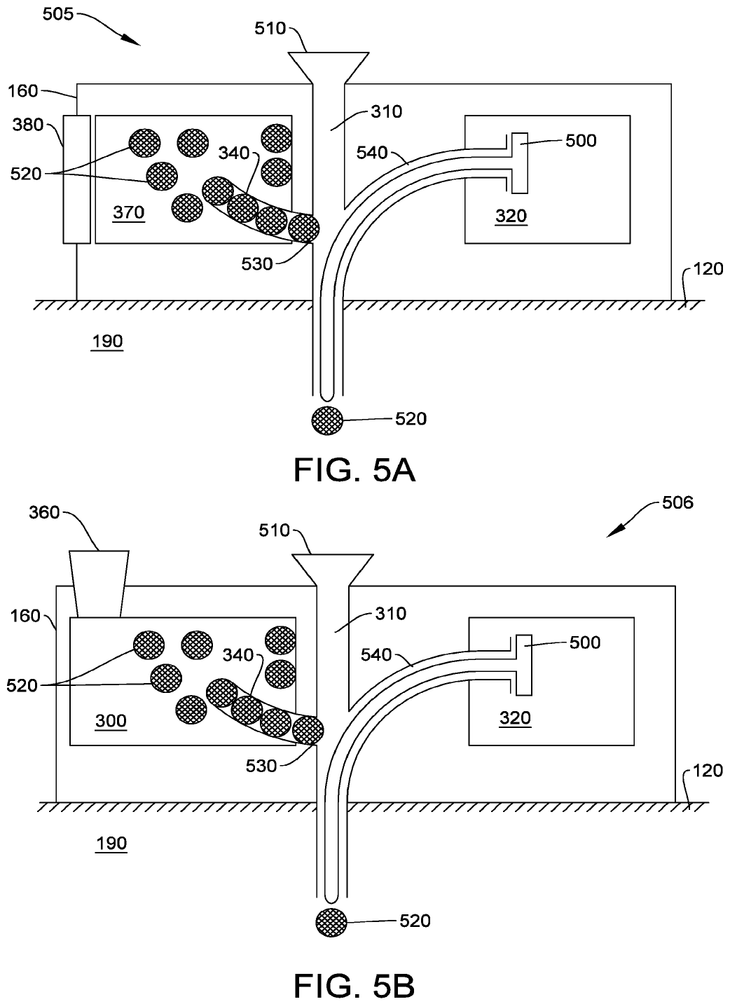

In certain embodiments, the formulation reservoir(s) is fillable and/or refillable while the delivery device is in place on the patient, either by direct introduction of solid therapeutic particles through a fill port on the housing in communication with the formulation reservoir (see FIG. 5B), or by replacement of a formulation reservoir “cartridge” that sits within the external housing (see FIG. 5A). The formulation reservoir cartridge allows for a “modular” configuration, where an entire formulation reservoir can be replaced. FIGS. 5A and 5B show two fillable/re-fillable configurations of the delivery device as shown in FIGS. 2A through 2D. FIG. 5A shows additional elements including a removable cartridge 370 containing solid therapeutic particles 520 in the external housing 160 of a delivery device 505. The removable cartridge 370 functions as a “modular” formulation reservoir, including a reservoir port 530. Reservoir port 530 of removable cartridge 370 may be kept sealed until placement into delivery device 505. A cassette exchange port or “door” 380 in housing 160 allows for exchange of formulation reservoir cartridges. FIG. 5B shows an expanded view of a delivery device 506 in which solid therapeutic particles are introduced into formulation reservoir 300 through a fill port 360, which is in communication with formulation reservoir 300 and preferably is self-sealing following the introduction of solid therapeutic particles. Fill port 360 can be used to fill or refill delivery device 506 as needed.

Due in part to the compact size of solid therapeutic particles, the delivery devices disclosed herein have a smaller dimension than traditional on-body pumps, e.g., insulin syringe pumps. The reduced dimensions of the disclosed delivery devices as compared to on-body liquid delivery devices currently of use in the art provide less bulk and less weight, allow for improved adherence to the skin, increase the locations on the body available for adherence of the device, and increase the amount of time a delivery device can remain in place. One example of such an on-body liquid device is approximately 3.9 cm×5.2 cm×1.45 cm and weighs approximately 25 grams. In some embodiments of the disclosed technology the on-body dispensing unit would be smaller and/or lighter than existing on-body liquid units. Additionally, embodiments of the delivery device allow a formulation reservoir containing the solid therapeutic particles to be refilled, so that the size of the formulation reservoir is not a limiting factor on how long the delivery device may remain in place on the patient.

In many embodiments, a processor-controller apparatus may be programmed to control the piston drive mechanism to provide automatic retraction and extension of the piston through the delivery channel and cannula at predetermined time intervals to deliver the solid therapeutic particles. Alternatively, the piston drive mechanism can also be user-controlled, where a user can activate the retraction and extension of the piston. In yet another alternative, the processor-controller apparatus may be programmed to provide automatic retraction and extension of the piston through the delivery channel and cannula at predetermined intervals to deliver the solid therapeutic particles to maintain a basal level of therapeutic agent in the body, but the processor-controller apparatus may be user-controlled to deliver, e.g., bolus doses of therapeutic agent. Additionally, in yet other embodiments, the delivery device may be entirely manual, without a power supply, processor-controller apparatus, or any other on-board electronics, resulting in a device requiring user input for delivery of solid therapeutic particles.

In one embodiment, the piston may be kept in an extended/deployed position between delivery events to prevent fluid from the patient's tissue from entering the delivery tube. In another embodiment, the cannula itself may be constructed of a collapsible material such as silicone that is expanded to allow release of solid therapeutic particles upon deployment of the piston into the delivery channel but otherwise is in a collapsed state that does not allow the flow of either solid therapeutic particles into the patient's tissue or of bodily fluids into the device.

In some embodiments, the delivery device may be placed on the skin of a patient using an introducer, including a sharp which is used to insert the cannula subcutaneously into the user. A sharp is typically formed using structurally rigid materials, such as metal or rigid plastic. Materials may include stainless steel and ABS (acrylonitrile-butadiene-styrene) plastic. In some embodiments, the sharp is pointed at the tip to facilitate penetration of the skin of the user. A thin sharp may reduce pain felt by the user upon insertion of the cannula.

In some embodiments, the delivery device may be introduced to the delivery site using a sharp and a septum for delivery of the cannula through the skin of the patient. When a sharp is used to introduce the on-body delivery device to the patient via a septum, the septum may be any shape that is compatible with such introduction, e.g., barrel-shaped, domed, flat or irregular in shape, as desired for a particular insertion application. One or more components of the introducer may be separate from the delivery device or the introducer and sharp may be integrated in the delivery device. Sharp systems and methods of use with the disclosed delivery devices include, but are not limited to, those shown in US Pub. Nos. 2015/0018639 to Stafford, et al.; 2008/0009692 to Stafford, et al.; 2015/0173661 to Myles, et al.; and 2015/0025345 to Funderburk et al., the disclosures of which are incorporated in full and for all purposes herein by reference.

In yet another embodiment, the delivery devices optionally may be integrated in a system with an on-body analyte sensor, where a patient wears both the delivery device and analyte sensing device on the body, where the on-body delivery device and the on-body analyte sensing device may be worn at different locations on the body. In certain embodiments, the analyte sensed may be the therapeutic agent itself. In other embodiments, the analyte sensed is an analyte that indicates a patient's response to a therapeutic agent, e.g., monitoring of glucose in response to the delivery of insulin. Examples of analytes that may be measured include for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined.

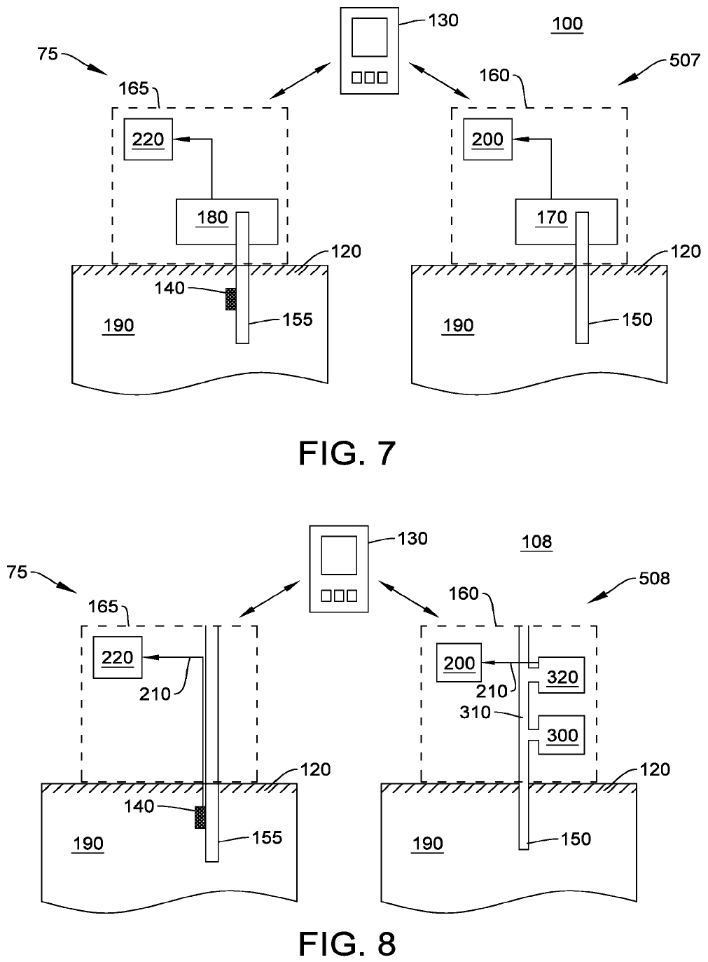

FIG. 7 shows one embodiment of an integrated system 100 in which a delivery device 507 delivers a solid therapeutic formulation by cannula 150, and an analyte sensing device 75 senses an analyte by a sensing element 140 on an insertable element 155. A processor-controller apparatus 200 controls solid therapeutic particle delivery according to, e.g., glucose levels sensed by analyte sensing device 75 and communicated via processor-controller apparatus 220 to reader 130. Exemplary on-body analyte sensing devices are disclosed, e.g., in U.S. Pat. No. 9,339,229 to Bernstein, the disclosure of which is incorporated in full and for all purposes herein by reference

FIG. 8 is a side view of an embodiment an integrated system 108. Integrated system 108 may include a delivery device 508 including components such as a formulation reservoir 300, a delivery tube 310, cannula 150, and a formulation insertion mechanism 320 as described in detail above. In an integrated system, sensing components are provided on an analyte sensing device 75, including a sensing element 140 that is positioned on an insertable element 155 in tissue 190 and operatively connected, electrically (e.g., by electrical wiring, conductive film, conductive traces, or other conductive material) or wirelessly, to processor-controller apparatus 220. A reader 130 receives data from processor-controller apparatus 220 of analyte sensing device 75 relating to an analyte concentration. A processor-controller apparatus (not shown) in reader 130 then communicates with the processor-controller apparatus 200 of delivery device 508 to dispense solid therapeutic particles. Optionally, a temperature measurement (or detection) device may be included on the analyte sensing device and is configured to monitor the temperature of the skin near the sensor insertion site. The temperature device is operatively coupled to processor-controller apparatus 220, and the user's temperature may be logged or both logged and displayed on the reader 130 or other device as described herein. The temperature reading may be used to adjust the analyte readings. Further, though FIGS. 7 and 8 illustrate embodiments of an integrated system where the delivery device and analyte sensing device are located in separate housings and may be placed at different places on the body, in some embodiments the delivery device and analyte sensing device may be contained within the same housing or housing components that are integrated on the same device.

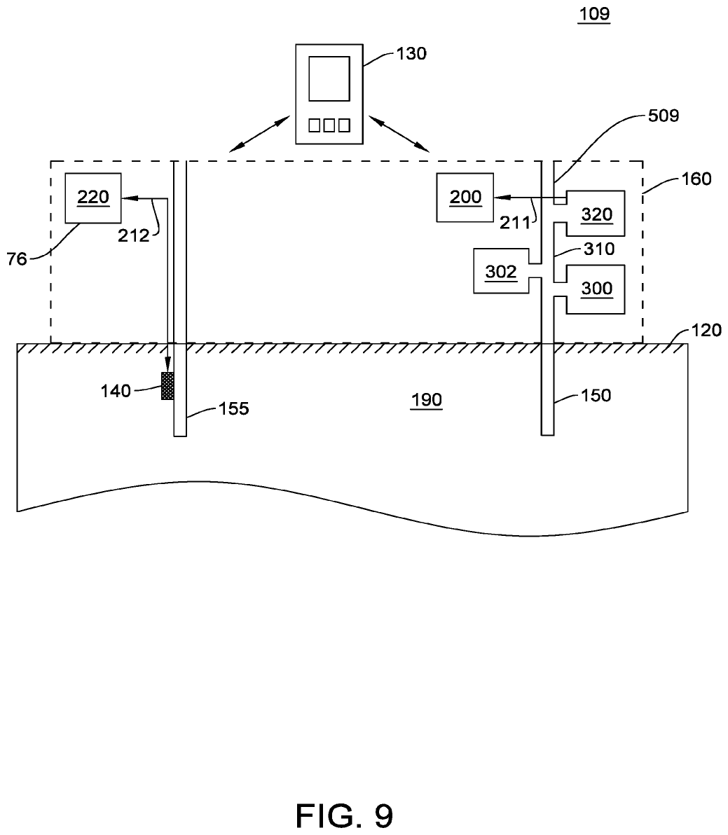

FIG. 9 is a side view of an integrated system 109 according to one embodiment of the disclosed technology. In this example, integrated system 109 may include a delivery device 509 and an analyte sensing device 76 in the same housing 160. The delivery device 509 may include components such as a first formulation reservoir 300, a second formulation reservoir 302, a delivery tube 310, cannula 150, and a formulation insertion mechanism 320. In this integrated system, sensing components are provided on an analyte sensing device 76, including a sensing element 140 that is positioned on an insertable element 155 in tissue 190 and operatively connected, electrically (e.g., by electrical wiring, conductive film, conductive traces, or other conductive material) or wirelessly, to processor-controller apparatus 220. A reader 130 receives data from processor-controller apparatus 220 of analyte sensing device 76 relating to an analyte concentration. A processor-controller apparatus (not shown) in reader 130 then communicates with the processor-controller apparatus 200 of delivery device 509 to dispense solid therapeutic particles. The processor-controller apparatus 200 of the delivery device 509 communicates (wirelessly or wired) with the formulation insertion mechanism 320 to initiate delivery or one or more therapeutic particles. Such particles may be delivered from either or both of the first formulation reservoir 300 and second formulation reservoir 302. Optionally, reader 130 includes a manual control feature which allows the user to manually select a desired number of therapeutic particles from either or both reservoirs 300, 302 and initiate their delivery by the formulation insertion mechanism 320.

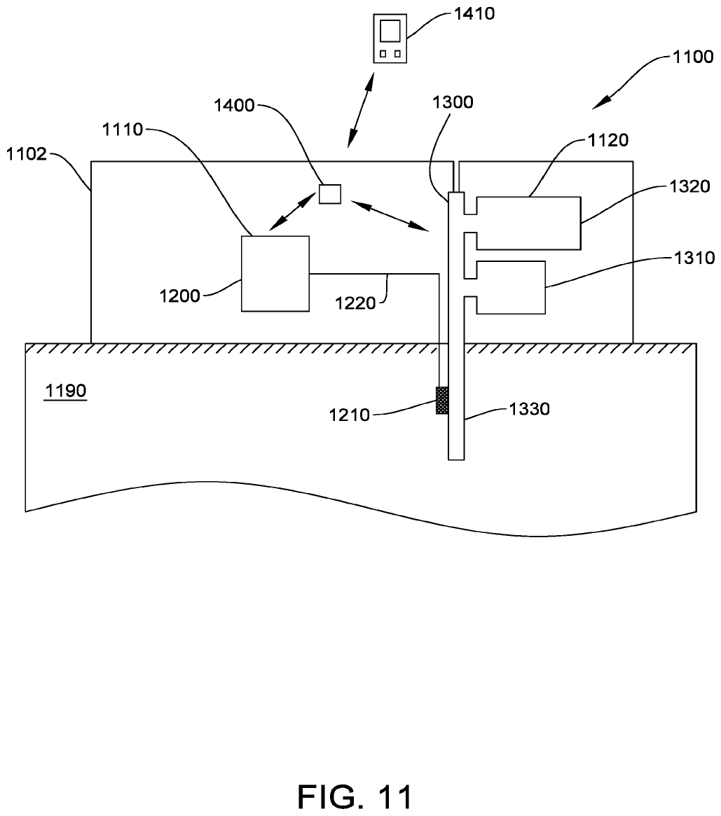

FIG. 11 is a side view of an embodiment of an integrated system 1100. Integrated system 1100 may include delivery device 1120 including components such as a formulation reservoir 1310, a delivery tube 1300, cannula 1330, and a formulation insertion mechanism 1320 as described in detail above. In this example integrated system, sensing components are provided on an analyte sensing device 1110, including a sensing element 1210 positioned on cannula 1330 and operatively connected, electrically (e.g., by electrical wiring, conductive film, conductive traces, or other conductive material) or wirelessly, to analyte-controller apparatus 1200 which is in communication with a processor-controller apparatus 1400. Optionally, an analyte controller may be integrated into a processor controller apparatus. A reader 1410 receives data from processor-controller apparatus 1400 of analyte sensing device 1110 relating to an analyte concentration. The reader 1410 then communicates with the processor-controller apparatus 1400 which instructs delivery device 1120 to dispense solid therapeutic particles. The delivery device 1120 components and sensing components may be contained in a housing 1102 which is mountable to a user's skin 1190 such as by an adhesive surface or other suitable attachment method.

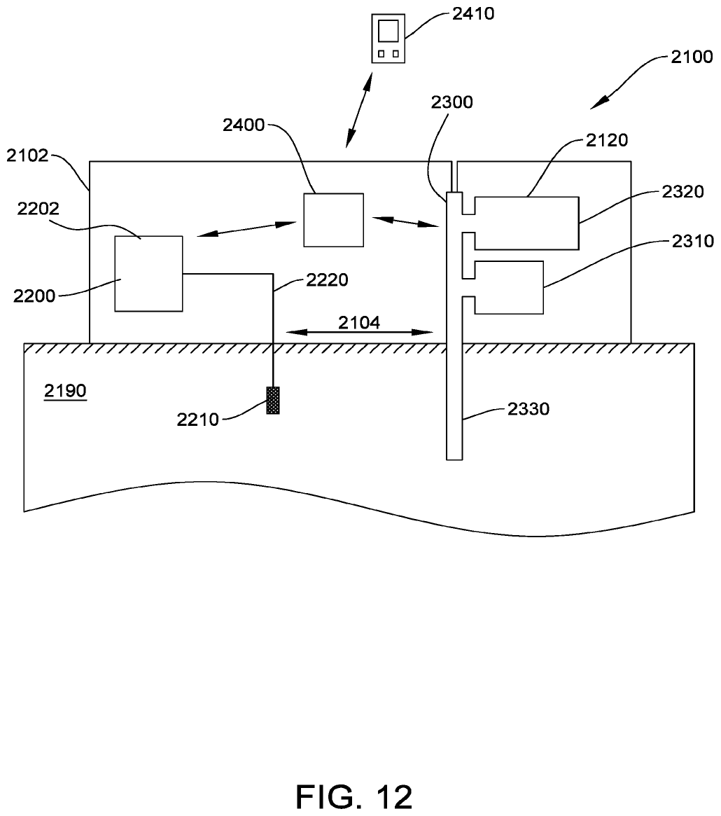

FIG. 12 is a side view of an embodiment of an integrated system 2100. Integrated system 2100 includes a delivery device 2120 and a sensing device 2202. Delivery device 2120 may include components such as a formulation reservoir 2310, a delivery tube 2300, cannula 2330, and a formulation insertion mechanism 2320 as such as those described in detail above. In this example integrated system 2100, sensing device components are provided on analyte sensing device 2202, including a sensing element 2210 operatively connected, electrically (e.g., by electrical wiring, conductive film, conductive traces, or other conductive material) or wirelessly, to analyte-controller apparatus 2200 which is in communication with a processor-controller apparatus 2400. In this example, sensing element is positioned at a distance 2104 from cannula 2330. Optionally, an analyte controller may be integrated into a processor controller apparatus. A reader 2410 receives data from processor-controller apparatus 2400 of analyte sensing device 2202 relating to an analyte concentration. The reader 2410 then communicates with the processor-controller apparatus 2400 which instructs delivery device 2120 to dispense solid therapeutic particles. The delivery device 2120 components and sensing components may be contained in a housing 2102 which is mountable to a user's skin 2190 such as by an adhesive surface or other suitable attachment method.

In another example, after receiving data from the processor-controller apparatus the reader generates a recommended dosing and communicates this information to the user, a physician, a medical professional, a remote computing device, or combinations thereof. Actual delivery of solid therapeutic particle(s) is then initiated manually by the user, physician, medical professional, or other person. Such manual initiation may optionally be accomplished using a reader, a remote device, or by directly interacting with the delivery device itself. In still other examples, the reader generates and communicates a recommended dosing to a user. If the user fails to input a response to this recommendation (such as a confirmation, modification, or cancellation) after a preset period of time the reader resends the recommendation, sends an alarm to the user and/or a third party such as a medical professional, and/or automatically initiates delivery of the recommended dose of therapeutic particle(s). In still other examples, the reader initiates automatic delivery of therapeutic particle(s) only under certain, predefined conditions, for example, if a condition such as hyperglycemia is detected.

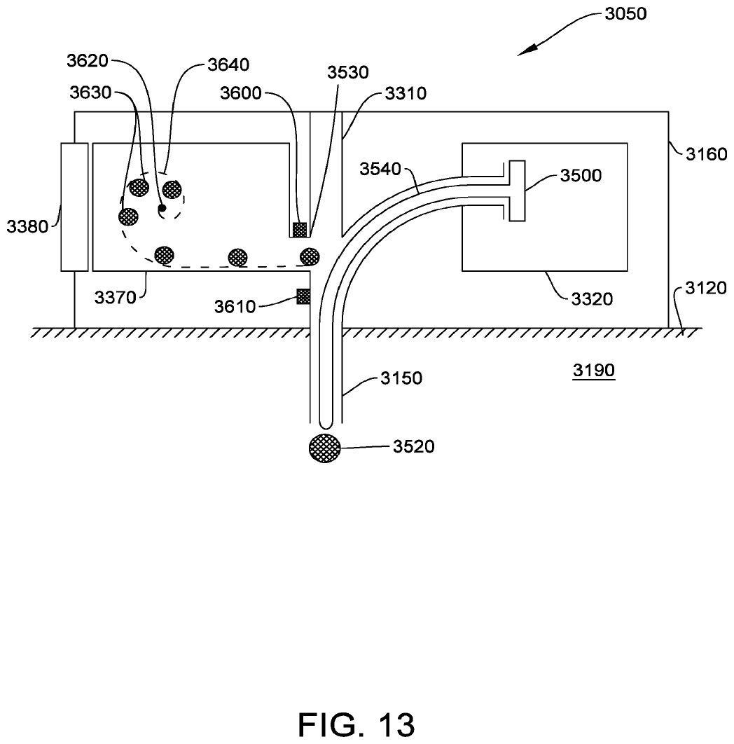

FIG. 13 is a cross sectional view of an embodiment of a delivery device 3050 having an external housing 3160 containing a formulation reservoir 3370, a delivery channel 3310 and a cannula 3150 that is placed into the subcutaneous tissue of a patient 3190. A proximal end of cannula 3150 is coupled to a distal end of a delivery channel 3310, and a distal end of cannula 3150 is inserted into the patient. In this example, cannula 3150 acts as a sharp to pierce the skin of the patient and position the cannula transcutaneously. In other examples, device 3050 further includes a septum (not shown) through which a sharp may be temporarily inserted to aid in insertion and positioning of the cannula.

In this example delivery device 3050 includes a removable cartridge 3370 containing solid therapeutic particles 3630 that is mountable in the external housing 3160 of a delivery device 3050. The removable cartridge 3370 functions as a “modular” formulation reservoir and includes a reservoir port 3530. Reservoir port 3530 of removable cartridge 3370 may be kept sealed until placement into delivery device 3050. A cassette exchange port or “door” 3380 in housing 3160 allows for exchange of formulation reservoir cartridges. The cartridge 3370 in this example includes a number of solid therapeutic particles 3630 disposed on dispensing ribbon or strip 3640 which is initially wound around an axle 3620.

When a therapeutic particle 3630 is to be dispensed in this example, the dispensing strip is unwound from the axle 3620 until a single therapeutic particle 3630 is disposed within delivery channel 3310. As piston 3500 is advanced by a piston drive mechanism 3320, it forces the therapeutic particle 3630 to separate from dispensing strip 3640, through the cannula 3150, and to be delivered to the subcutaneous tissue 3190 of the patient. Optionally, the dispensing strip is made from an inert material which dissolves when inserted into the patient along with a therapeutic particle. The positioning of piston 3500 is controlled by a piston drive mechanism 3320 in engagement with piston 3500 and controlled by a processor-controller apparatus (not shown).

Continuing with the example shown in FIG. 13, delivery device may also include one or more detection mechanisms to insure delivery of the correct dose of therapeutic particles. Such detection mechanisms may be optical, electrical, or mechanical in nature. In this example, a mechanical detector 3600 is positioned so as to detect the passage of a therapeutic particle through the reservoir port 3530 and into the delivery channel 3310. An optical sensor 3610 positioned in the delivery channel 3310 detects passage of a therapeutic particle through the delivery channel 3310 and into the cannula 3150.

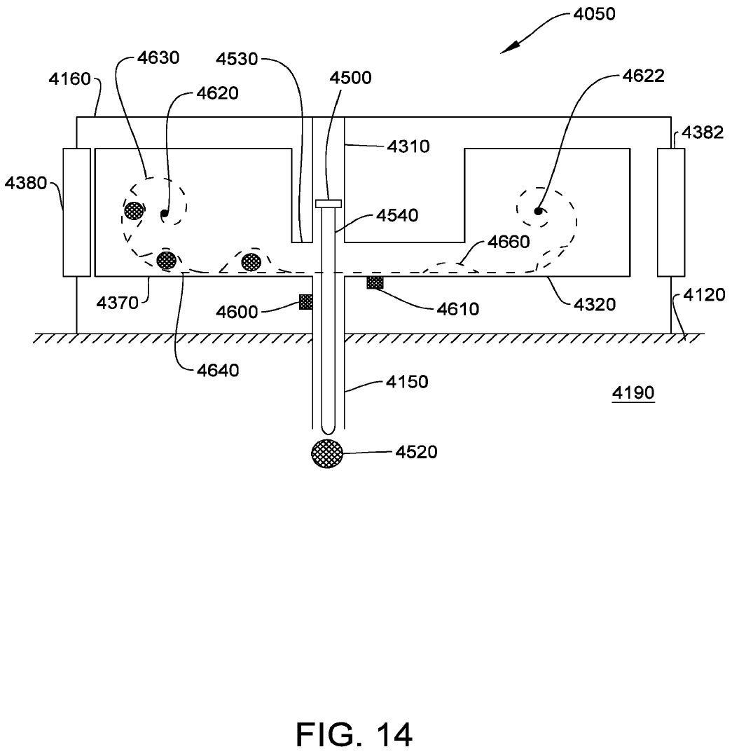

FIG. 14 is a cross sectional view of an embodiment of a delivery device 4050 having an external housing 4160 containing a formulation reservoir 4370, a delivery channel 4310 and a cannula 4150 that is placed into the subcutaneous tissue 4120 of a patient. A proximal end of cannula 4150 is coupled to a distal end of a delivery channel 4310, and a distal end of cannula 4150 is inserted into the patient. In this example, cannula 4150 acts as a sharp to pierce the skin of the patient and position the cannula transcutaneously. In other examples, device 4050 further includes a septum (not shown) through which a sharp may be temporarily inserted to aid in insertion and positioning of the cannula.

In this example delivery device 4050 includes a removable cartridge 4370 containing solid therapeutic particles 4640 is mountable in the external housing 4160 configured for mounting to the skin 4120 of a patient. The removable cartridge 4370 functions as a “modular” formulation reservoir, and includes a reservoir port 4530. Reservoir port 4530 of removable cartridge 4370 may be kept sealed until placement into delivery device 4050. A cassette exchange port 4380 in housing 4160 allows for exchange of formulation reservoir cartridges 4370. The cartridge 4370 in this example includes a number of solid therapeutic particles 4640 disposed on dispensing ribbon or strip 4630 which is initially wound around an axle 4620. The dispensing ribbon 4630 is fed into a second cartridge or housing 4320 and wound around an axle 4622.

When a therapeutic particle 4640 is to be dispensed in this example, the dispensing strip 4630 is unwound from the axle 4620 until a single therapeutic particle 4640 is disposed within delivery channel 4310. As piston 4500 is advanced by a piston drive mechanism (not shown), it forces the therapeutic particle 4640 to be ejected from a capsule or blister 4650 in dispensing strip 4630, through the cannula 4150, and to be delivered to the subcutaneous tissue 4190 of the patient. The emptied blister 4660 on dispensing ribbon 4630 is advanced into housing 4320 and wound around an axle 4622. Once dispensing ribbon 4630 is empty it may be removed from housing 4320 through an opening or port 4382 in the housing 4320.

Optionally, all or part of the dispensing strip is made from an inert material which dissolves when inserted into the patient along with a therapeutic particle. The positioning of piston 4500 is controlled by a piston drive mechanism (not shown) in engagement with piston 4500 and controlled by a processor-controller apparatus (not shown). The piston drive mechanism may be driven an electric, electro-mechanical, pneumatic, or other suitable means.

Continuing with the example shown in FIG. 14, delivery device 4050 may also include one or more detection mechanisms to insure delivery of the correct dose of therapeutic particles. Such detection mechanisms may be optical, electrical, or mechanical in nature. In this example, a detector 4600 is positioned so as to detect the passage of a therapeutic particle through the cannula 4150 and into the patient's tissue 4190. For example, if the detector 4600 is an optical sensor it may detect the passage of a therapeutic particle as a shadow or other interruption of light prior to the passage of the piston. A sensor 4610 positioned in housing 4320 may detect passage of an emptied blister 4660 on dispensing ribbon 4630. For example, if detector 4610 is an optical detector and dispensing ribbon 4630 is optically transparent, an interruption or shadow passing over detector 4610 would indicate that a therapeutic particle had failed to be properly ejected from the strip and inserted into the patient. A mechanical detector 4610 might detect a full blister 4650 as increased thickness along ribbon 4630 as it passes.

The analyte sensing device may be configured to require no system calibration or no user calibration. For example, a sensing element may be factory calibrated and not require further calibrating such as the system described in US Pub. No. 2016/0000360 to Feldman, the disclosure of which is incorporated in full and for all purposes herein by reference.

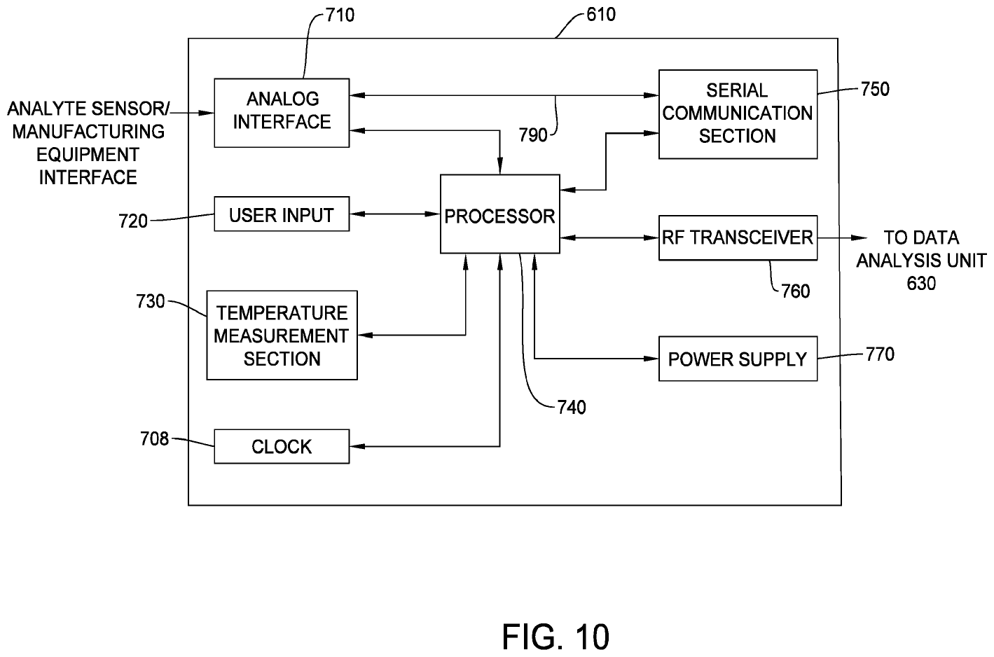

FIG. 10 is a block diagram of the data processing unit of the processor-controller apparatus 610 in accordance with an embodiment of the disclosed technology where the delivery device is one component in an integrated system that also includes an on-body analyte sensing device. Examples of such processor-controller devices may be integrated into a solid formulation delivery device according to the disclosed technology (with or without an integrated analyte sensor) or be separate units. Stand alone processor-controller devices may be designed to be worn on-body or hand held, as desired. The processor-controller apparatus 610 in one embodiment includes an analog interface 710 configured to communicate with a sensing element 140 (such as shown in FIG. 7), a user input 720, and, optionally, a temperature detection section 730 (such as described in relation to FIG. 8), each of which is operatively coupled to a data processing unit processor 740 such as one or more central processing units (CPUs) or equivalent microprocessor units. Further shown in FIG. 10 are a transmitter serial communication section 750 and an RF transceiver 760, each of which is also operatively coupled to processor. In one embodiment, serial communication section 750 may be operatively coupled to the analog interface 710 via communication link 790. Moreover, a power supply 770 such as a battery is also provided in processor-controller apparatus 610 to provide the necessary power for the components in processor-controller apparatus 610. Additionally, as can be seen from the figure, clock 708 is provided to, among others, supply real time information to processor. Optionally, a processor-controller apparatus may include a memory unit for storing instructions to be run by the processor and/or recording operational data from a solid formulation delivery device and/or analyte sensor.

Additional detailed descriptions of the data monitoring and management systems, such as analyte monitoring systems, and the various components including the functional descriptions of the processor-controller apparatus are provided in U.S. Pat. No. 6,175,752 to Say et al., and U.S. Pat. No. 7,811,231 to Jin et al., the disclosures of which are incorporated in full and for all purposes herein by reference.

FIG. 6 shows an example of an integrated system according to the disclosed technology. In this example, an integrated system includes an on-body delivery device 1000, which optionally includes an analyte sensor, and a data processing module 1020, which may optionally be integrated into the housing of the on-body delivery device 1000. The data processing module 1020 may communicate with a remote terminal 1040 such as a personal computer, a laptop computer, a tablet, or other suitable data processing device capable of running software for data management, analysis, and communication with the components of the integrated system. The data processing module may also communicate with a display device/controller 1030 such as a smartphone or other suitable device capable of running software for data management, analysis, and communication with the components of the integrated system. The display device/controller 1030, the remote terminal, and/or the data processing module 1020 may all be capable of communicating with each other using direct, wired connections, or suitable wireless connections (Wi-Fi, RFID, Bluetooth, and the like), and all may also be capable of communication with remote data storage/computing system 1050 including individual servers or multiple remote servers (e.g., cloud storage).

It should be understood that the disclosed technology is described primarily with respect to an insulin delivery device and a glucose monitoring system for convenience; however, such description is in no way intended to limit the scope of the disclosed technology. Also, it is to be understood that an integrated system incorporating the disclosed technology may be configured to monitor a variety of analytes, e.g., lactate, and the like, and to dispense solid therapeutic particles of any appropriate therapeutic agent. In addition, the devices and the components thereof in the Figures are not necessarily drawn to scale, but instead are illustrated to further understanding of the disclosed delivery devices and integrated systems.

The implementations set forth in the foregoing description do not represent all implementations consistent with the subject matter described herein. Instead, they are merely some examples consistent with aspects related to the described subject matter. Other aspects, advantages, and modifications are considered to be within the scope of the claims presented below. The claims presented are representative of the subject matter disclosed herein. Other, unclaimed aspects of the disclosed subject matter are also contemplated.

Wherever possible, the same reference numbers have been used throughout the drawings to refer to the same or like parts. Although a few variations have been described in detail above, other modifications or additions are possible. In particular, further features and/or variations may be provided in addition to those set forth herein. For example, the implementations described above may be directed to various combinations and sub-combinations of the disclosed features and/or combinations and sub-combinations of several further features disclosed above. In addition, the logic flow depicted in the accompanying figures and/or described herein does not require the particular order shown, or sequential order, to achieve desirable results. Other embodiments may be within the scope of the following claims. All references cited herein are hereby incorporated by reference into the detailed description for all purposes.

1. An on-body device for delivery of solid therapeutic particles to a patient, comprising:
a housing having a mounting surface for mounting the device to the patient;
a formulation reservoir disposed within the housing for storage of solid therapeutic particles;
a delivery channel disposed within the housing and configured to accept solid therapeutic particles from the formulation reservoir;
a cannula coupled to the distal end of the delivery channel and configured for placement in the patient's subcutaneous tissue; and
a transfer mechanism for advancing one or more individual solid therapeutic particles from the delivery channel through the cannula and into the patient.

2. The device of claim 1, further comprising a particle distribution mechanism disposed within the formulation reservoir.

3. The device of claim 1, wherein the transfer mechanism prevents dispensed solid therapeutic particles from moving back into the cannula.

4. The device of claim 1, wherein the delivery channel further includes a septum at one end.

5. The device of claim 1, wherein the formulation reservoir is refillable and wherein the formulation reservoir may be refilled without removal of the device from the patient.

6. (canceled)

7. The device of claim 1, wherein the formulation reservoir is removable and replaceable, and wherein the formulation reservoir may be replaced in the housing without removal of the device from the patient.

8. (canceled)

9. The device of claim 1, further comprising a particle detection mechanism, and wherein the detection mechanism is optical or mechanical.

10.-11. (canceled)

12. The device of claim 1, wherein the solid therapeutic particles comprise insulin or an analog thereof.

13. The device of claim 1, wherein the device is manually operated or is configured to automatically deliver the solid therapeutic particles to the patient.

14. (canceled)

15. The device of claim 1, wherein the cannula is collapsible.

16. (canceled)

17. The device of claim 1, wherein the device is part of an integrated system, and the integrated system further comprises a processor-controller apparatus.

18. The device of claim 17, wherein the integrated system further comprises an analyte sensing device in communication with the processor-controller apparatus.

19-38. (canceled)

39. A method of delivering solid therapeutic particles to a patient, comprising:
mounting an on-body delivery device on a skin surface of the patient, the device comprising:
a housing having a mounting surface for mounting the device to the patient;
a formulation reservoir disposed within the housing for storage of solid therapeutic particles;
a delivery channel disposed within the housing and configured to accept solid therapeutic particles from the formulation reservoir;
a cannula coupled to the distal end of the delivery channel and configured for placement in the patient's subcutaneous tissue; and
a transfer mechanism for advancing one or more individual solid therapeutic particles from the delivery channel through the cannula and into the patient;
transferring at least one solid therapeutic particle from the formulation reservoir to the delivery channel; and
delivering, using the transfer mechanism, the at least one solid therapeutic particle from the delivery channel through the cannula into the patient's subcutaneous tissue.

40. The method of claim 39, wherein the delivery of the at least one solid therapeutic particle occurs automatically without any input from the patient.

41. The method of claim 39, further comprising:
providing an analyte sensing device with a sensing element in fluid contact with a bodily fluid of the patient;
detecting an analyte level of the patient using the analyte sensing device; and
determining a recommended dose of solid therapeutic particles based at least in part on the detected analyte level.

42. The method of claim 41, wherein the on-body delivery device and the analyte sensing device are located in separate housings, or wherein the on-body delivery device and the analyte sensing device are located in the same housing or set of integrated housing components.

43. (canceled)

44. The method of claim 41, further comprising:
communicating the dosage recommendation to the patient;
receiving input from the patient; and
delivering, using the transfer mechanism, the at least one solid therapeutic particle in response to the patient input.

45. The method of claim 44, wherein the patient input is provided by interaction with the on-body delivery device.

46. The method of claim 44, wherein the patient input is provided by interaction with a remote device in communication with the on-body delivery device.

47. The method of claim 41, further comprising:
communicating the dosage recommendation to the patient; and
if no patient input is received within a predetermined period of time after the dosage recommendation is communicated, automatically initiating delivery of the recommended dose of solid therapeutic particles, or communicating an alarm to at least one of the patient and a third party.

48-191. (canceled)
